(19) United States
(12) Patent Application Publication
(10) Pub. No.: US 2010/0008897 A1
(43) Pub. Date: Jan. 14, 2010

(54) COMPOSITION FOR PROVIDING A BENEFIT TO A KERATIN-CONTAINING SUBSTRATE

(76) Inventors: Susan Daly, Basking Ridge, NJ (US); Janusz Jachowicz, Bethel, CT (US); Robert Bianchini, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/170,007

(22) Filed: Jul. 9, 2008

(51) Int. Cl. A61K 38/43; A61K 38/02; A61K 38/47; A61K 38/48

(52) U.S. Cl. 424/94.61; 514/12; 424/94.1; 424/94.63

(57) ABSTRACT

This invention relates to compositions and methods for providing cosmetic and other potential benefits to keratin-containing substrates. The compositions contain at least one naturally-occurring cationic protein with a biological function in a cosmetically acceptable carrier, such that the naturally-occurring cationic protein retains its essential configuration, preserving its biological function, when it is in proximity to a keratin-containing substrate.

### FIELD OF THE INVENTION

This invention relates to compositions and methods for providing cosmetic benefits to keratin-containing substrates, and more particularly to hair treatment compositions containing naturally-occurring cationic proteins and methods of using them.

### BACKGROUND OF THE INVENTION

Consumers use a variety of hair care and treatment products to obtain various desired cosmetic effects on their hair. These products may be used, for example, for cleansing, conditioning (i.e., imparting shine, softness, manageability, and ease of combing), thickening, hair repair, protection from environmental factors, and to achieve other cosmetic benefits. These effects may be temporary such that they are removed by rinsing with water or shampooing, or longer lasting such that they remain after rinsing or shampooing.

One potential desired benefit is the creation of an antimicrobial effect on hair. Such an effect would be particularly beneficial for people with oily or acne-prone skin. Often, people with acne-prone skin are advised to keep their hands and hair away from their faces to prevent microbes on their hands and hair from contacting their facial skin and thus, contributing to acne outbreaks. Antimicrobial hair fibers would also be beneficial in antimicrobial makeup brushes and for underarm anti-odor protection.

A problem with currently available antimicrobial compounds is that they do not substantially deposit onto hair or skin or other keratin-containing substrates. Most of these currently available antimicrobials demonstrate antimicrobial activity in a solution or in a formulation. However, when these solutions or formulations are applied to hair or skin, and then rinsed with water or washed with soap or shampoo, the antimicrobial compounds immediately wash away, with little or no active compound remaining on the hair or skin.

It may also be desired to treat keratin-containing substrates with natural proteins to impart various benefits. However, many of these proteins do not significantly deposit onto hair surfaces. Protein hydrolysates that are often used in hair care products, such as soy, wheat, silk, and keratin hydrolysates, are among those that do not significantly deposit onto hair, even at high concentrations in the formulation (>0.5 wt. %), unless cysteine is a major component of the peptide fragment.

Surprisingly, we have found an effective method of depositing proteins, such as antimicrobially active proteins, onto hair and other keratin-containing surfaces such that the cosmetic effects of the proteins remain even after rinsing with water or washing with soap or shampoo.

### SUMMARY OF THE INVENTION

This invention relates to a cosmetic composition for providing a cosmetic benefit to a keratin-containing substrate, containing the following components:
a) at least one naturally-occurring cationic protein having a biological function; and
b) a cosmetically acceptable carrier;
   wherein the naturally-occurring cationic protein retains its essential configuration when in proximity to the keratin-containing substrate to preserve its biological function.

Additionally, this invention relates to methods for providing cosmetic benefits to keratin-containing substrates. The method involves
a) providing a cosmetic composition comprising at least one naturally-occurring cationic protein having a biological function; and a cosmetically acceptable carrier;
b) applying the cosmetic composition to the keratin-containing substrate for a time period sufficient for the naturally-occurring cationic protein to be deposited on the substrate and form a layer; and
c) rinsing the cosmetic composition from the substrate with water;
   wherein the naturally-occurring cationic protein retains its essential configuration when in proximity to the keratin-containing substrate to preserve the biological function.

Other features and advantages of this invention will be apparent from the detailed description of the invention and from the claims.

### BRIEF DESCRIPTION OF THE DRAWINGS

FIG. 1 is a chart showing the fluorescence intensity and the delta streaming potential as a function of protein concentration, illustrating the results obtained in Example 3.

FIG. 2 is a chart showing the streaming potential as a function of time, illustrating the results obtained in Example 4.

### DETAILED DESCRIPTION OF THE INVENTION

The compositions of this invention contain naturally-occurring, biologically functional, cationic proteins which unexpectedly retain their biological functions when in proximity to keratin-containing substrates.

Naturally-occurring proteins perform a wide variety of biological functions, including antimicrobial activity, which may be of benefit in cosmetic compositions for keratin-containing substrates, such as hair, skin, nails, teeth, mucosa, tissues, wool, and fur, among others. The biological functionalities of naturally-occurring proteins are generally dependent upon their essential configurations. However, a number of factors may work to alter these configurations, causing loss of the respective functionalities. These factors include, among others, protein aggregation, denaturation, and stereohindrance.

For example, the naturally-occurring cationic protein lysozyme demonstrates an antimicrobial function: the lysozyme breaks down the polysaccharide cell wall of some kinds of bacteria. This action is accomplished largely because of the stereochemistry of the lysozyme molecule, which is generally globular in shape with a deep indentation. Because this indentation plays an important role in the mechanism of bacterial cell wall breakdown, stereohindrance of the lysozyme molecule could alter the conformation of lysozyme molecue by changing its indentation such that the molecule's antibacterial activity may be rendered nonfunctional.

Most keratin-containing substrates are anionically charged. Thus, application of naturally-occurring functional proteins to such substrates might be expected to result in failure of the proteins to deposit on the substrates. If such proteins are able to be deposited on the substrate, it might be expected that changes to the protein molecules (i.e., stereohindrance, denaturation, aggregation) could result such that their respective functionalities would be inactivated. Surprisingly, though, the compositions and methods of this invention containing at least one naturally-occurring functional cationic protein have been observed to deposit protein onto keratin-containing substrates, specifically hair, enabling the protein also to retain its essential configuration and its biological functionality.

It is believed that one skilled in the art can, based upon the description herein, utilize the compositions and methods of this invention to their fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

### DEFINITIONS

“Keratin-containing substrate”, as used herein, includes hair, skin, nails, teeth, tissues, wool, fur, and any other materials that contain keratin proteins. Keratin-containing substrate of this invention is preferably human hair, skin, or nail.

“Cationic compound”, as used herein, relates to a compound with a positive charge. Such compounds generally move toward the negative electrode in electrolysis.

“Anionic compound”, as used herein, relates to a compound with a negative charge. Such compounds generally move toward the positive electrode in electrolysis.

“Peptide”, as used herein, is a molecule containing two or more amino acids joined by a peptide bond or modified peptide bonds.

The term “amino acid” refers to the basic chemical structural unit of a protein or polypeptide. The following abbreviations are used herein to identify specific amino acids:

TABLE 1

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Protein”, as used herein, relates to a long chain of amino acids joined together by peptide bonds. Proteins may generally have molecular weights more than 10,000.

“Naturally-occurring”, as used herein, relates to compounds that occur in nature without human intervention. It may also relate to compounds that are synthesized by humans to be identical to those that occur in nature.

“Biological function”, as used herein, refers to any type of natural protein activity. Nonlimiting examples of biological function include enzymatic activity, antibody binding, protein binding to a target molecule (such as histone binding to DNA), antifungal activity, antimicrobial activity, growth promotion and regulation, receptor activity, vitamin binding, digestion, respiration, inflammation, anti-inflammation, and the like.

“Essential configuration”, as used herein, refers to the molecular organization of the protein at the hair surface. This molecular organization may include the protein's conformation or secondary structure, the protein's orientation relative to the hair surface, accessibility of the protein's active site to a protein substrate, and aggregation or clustering of the protein on the surface. All of these organizational factors can result in decreased biological functional properties of the protein. The protein's “active site” is the location on the protein responsible for its biological function. The “protein substrate” is another molecule that is acted upon by the protein's active site.

“Isoelectric Point” or “IEP” or “pI”, as used herein, refers to the pH value at which a substance, compound, molecule, or surface carries no net electrical charge or shows no migration under the influence of an electric field.

### Naturally-Occurring Cationic Proteins

Examples of naturally-occurring cationic proteins for use in this invention include, without limitation, lysozyme; biotin-binding proteins, including avidin; antimicrobial proteins, including magainin, defensins, cathelicdin; RNA or DNA binding proteins, including histones, ribonuclease A, deoxyribonuclease (DNase); proteases, including trypsin, chymotrypsin, papain, caspase; methylated collagen; cytochrome C; proteins involved in the aging process, including telomerase; platelet factor 4; and protamine sulfate.

Preferred for use in this invention are naturally-occurring cationic proteins with an isoelectric point of greater than 6, preferably between 8-12, or in other words, proteins that are positively charged when incorporated in a formulation of pH≧5.

Preferred concentrations of naturally-occurring cationic proteins in the compositions and methods of this invention range from about 0.000001% to about 10% by weight. More preferably, the concentration ranges from about 0.001% to about 5% by weight. Even more preferably, the concentration ranges from about 0.01% to about 2% by weight.

A naturally-occurring cationic protein which is preferred for use in an embodiment of this invention is lysozyme, which demonstrates an antimicrobial effect. The lysozyme adsorbs onto hair while retaining its antimicrobial properties when applied to the hair in compositions and according to methods of this invention. Lysozyme has a molecular weight of about 14.6 kDa. An advantage to using lysozyme for its antimicrobial properties is its relatively large molecule size. Antimicrobials with smaller molecule size can penetrate the skin and thus, are more likely to cause skin irritation to the consumer.

### Cosmetically Acceptable Carrier

The compositions of this invention contain one or more cosmetically-acceptable carriers. Preferably, such carriers include water. Organic solvents may also be included in order to facilitate manufacturing of the compositions or to provide esthetic properties, such as viscosity control, as long as the biological function of the naturally-occurring protein is retained. Suitable solvents include the lower alcohols, or C2-C6 alcohols, such as ethanol, propanol, isopropanol, butanols, pentanols, and hexanols; glycol ethers, such as 2-butoxyethanol, ethylene glycol monoethyl ether, propylene glycol and diethylene glycol monoethyl ether or monomethyl ether; and the mixtures thereof. A preferred organic solvent in this invention is ethanol.

### Other Cosmetic Additives

In addition to the above-described ingredients, other common cosmetic components and additives known or otherwise effective for use in hair care or personal care products may be incorporated in the compositions of this invention, as long as the basic properties of the compositions, and the biological function of the naturally-occurring cationic protein, are not adversely affected. Such optional ingredients include, but are not limited to, anti-dandruff agents, hair growth agents, anti-inflammatory agents, anti-microbial agents, anionic and nonionic surfactants, suspending agents, humectants, emollients, moisturizers, fragrances, dyes and colorants, foam stabilizers, anti-static agents, preservatives, rheology modifiers, water softening agents, chelants, hydrotropes, polyalkylene glycols, acids, bases, buffers, beads, pearlescent aids, fatty alcohols, proteins, skin active agents, sunscreens, vitamins, and pediculocides, and the like. Optional components may be present in weight percentages of less than about 1% each, and from about 0.01% to about 10% by weight of the composition in total.

The compositions of this invention should be stable to phase or ingredient separation at a temperature of about 25° C. for a long period of time, or at least for about 26 weeks at a temperature of between 4° C. and 40° C. Thus, the compositions of this invention have demonstrated sufficient stability to phase and ingredient separation at temperatures normally found in commercial product storage and shipping to remain unaffected for a period of at least six months.

This invention also relates to methods of using the compositions of this invention to provide cosmetic benefits to keratin-containing substrates, including hair. Although the following recites hair as the substrate to which the benefits are to be imparted, the method described herein may be applied to other keratin-containing substrates that are amenable to being treated with cosmetically beneficial compositions containing naturally-occurring cationic proteins such as are described in this invention. Treatment of hair with the compositions of this invention is generally carried out by: (1) applying to wet or dry hair a sufficient amount of the cosmetically beneficial composition according to the invention; (2) distributing the composition according to this invention more or less evenly throughout the hair such that it contacts all the hair or other substrate which is intended to be treated. This permits the naturally-occurring cationic proteins of the compositions of this invention to deposit onto the surface of the hair or other keratin-containing substrate. This distribution step may be accomplished by rubbing the composition throughout the hair or onto the substrate manually or using a hair appliance such as a comb or a brush; (3) allowing the composition of the invention to remain on the hair or substrate for about 1 to about 20 minutes, more preferably from about 1 to about 10 minutes, and even more preferably from about 2 to about 5 minutes; and (4) optionally, rinsing said hair or other substrate with water so as to remove excess material that has not adsorbed onto the hair or substrate.

### Cosmetic Benefits

The compositions and methods of this invention may be formulated to provide any cosmetic benefit to a keratin-containing substrate, preferably human hair, skin, or nails. Preferred cosmetic benefits addressed and provided by compositions and methods of this invention include anti-aging, antimicrobial, antifungal, anti-inflammation, coloring, skin tightening, conditioning, cleansing, and combinations thereof.

### Streaming Potential

Streaming potential is an electrokinetic measurement determined by passing an electrolytic solution through a permeable body, such as a capillary, a porous solid, or a plug of fiber such as hair. The streaming of the liquid through the permeable body produces an electrokinetic potential that may be measured. An electrometer may be used to measure the electrical potential across the plug caused by the flow of liquid. A detailed description of streaming potential can be found in U.S. Pat. No. 5,452,233.

In methods and compositions of this invention, streaming potential (E) is used to measure the surface charge on hair before and after treatments with certain compounds. Any change in the streaming potential after treatment indicates a change in the surface charge of the hair, and thus the streaming potential measurement may be used to monitor the deposition and retention of the treatment compounds on the hair. This change in streaming potential, or delta streaming potential, or ΔE, is calculated as

ΔE=E_{after treatment}−E_{before treatmet}.

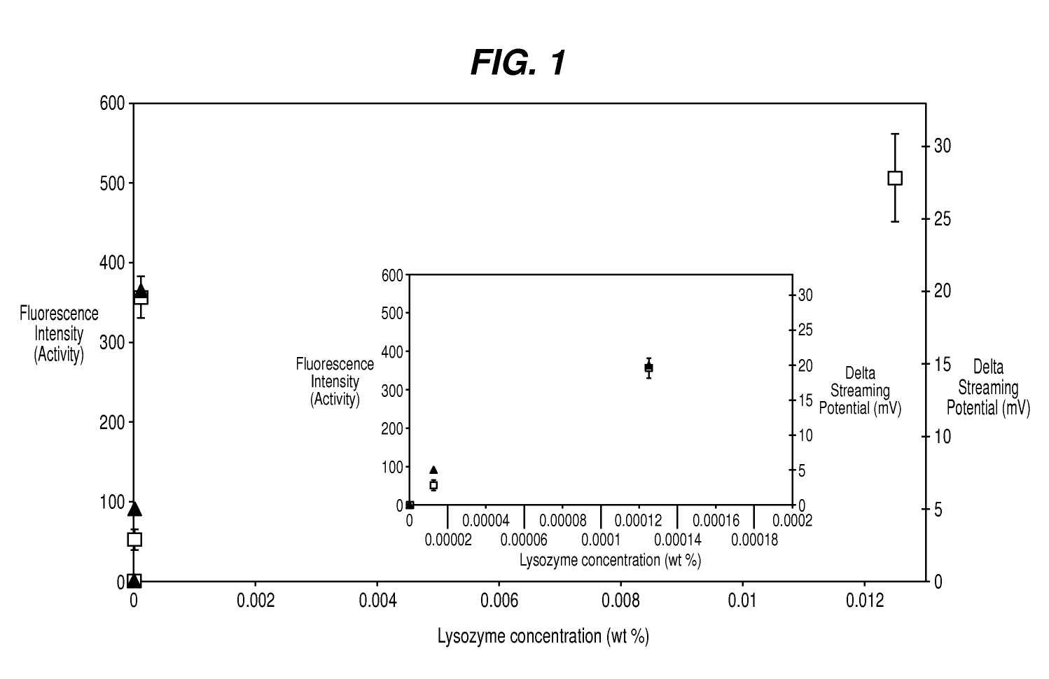

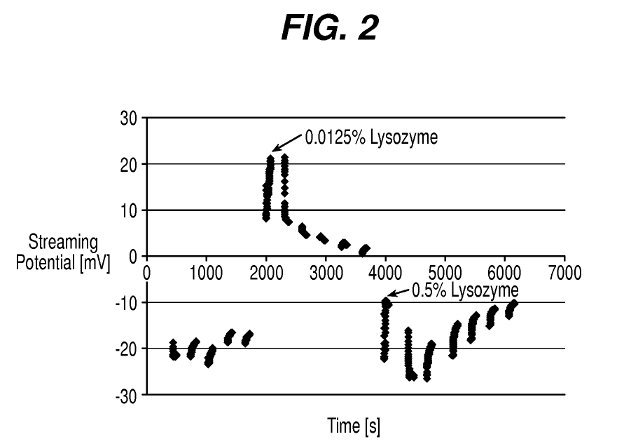

In FIG. 1, the streaming potential measurement is illustrated as a graph where the x-axis represents lysozyme concentration in wt. %, and the y-axis on the right side represents delta streaming potential between untreated hair and lysozyme-treated hair. In FIG. 2, the streaming potential measurement is illustrated as a graph where the x-axis represents time, measured in seconds in this invention, and the y-axis represents the streaming potential, measured in millivolts (mV) in this invention.

The human hair used in the examples below was blonde hair. Such hair is available commercially, for example from International Hair Importers and Products (Bellerose, N.Y.), and is also available in different colors, such as brown, black, red, and blonde, and in various types, such as African-American, Caucasian, and Asian.

The lysozyme from chicken egg white used in the examples below was obtained from Sigma Aldrich Company of St. Louis, Mo.

### Lysozyme Activity Assay

Lysozyme antimicrobial activity may be measured spectroscopically, using a Lysozyme Assay Kit such as that sold commercially as E-22013 by Molecular Probes, Inc. of Eugene, Oreg. In general, the assay involves incubating various concentrations of lysozyme in a buffered solution with Micrococcus lysodeikticus cells which have been labeled with fluorescein to such a degree that the fluorescence is quenched. As the lysozyme acts upon the microbes, the quenching is relieved, and the fluorescence of the solution increases. The fluorescence increase can then be measured using any spectrofluorometer that can detect fluorescein. The fluorescence intensity measurement of this assay was used to demonstrate lysozyme activity in the examples of this invention, and is shown by the black triangles and indicated on the left side of the y-axis of FIG. 1 as fluorescence units.

### Zeta Potential

Zeta potential is the average potential in the hydrodynamic plane of shear, separating the bulk liquid phase and the diffuse layers of the electrochemical double layer, and can be calculated from the streaming potential or streaming current measurement.

### EXAMPLE 1

Streaming potential analysis was conducted on blonde hair showing the effect on streaming potential of untreated hair and of various concentrations of lysozyme when applied to the hair. All treatment solutions were made with the specified concentration of lysozyme in a solution 1 mM KCl in deionized water. The results are shown in Table 1 below.

TABLE 1

| Concentration Lysozyme | Streaming potential E | Delta streaming |
|---|---|---|
| (Weight %) | (mV) | potential ΔE (mV) |
| 0.000000 | −22 | — |
| (untreated hair) | | |
| 0.00000625 | −18 | 4 |
| 0.0000625 | −2 | 20 |
| 0.000625 | 5 | 27 |
| 0.0125 | 7 | 29 |

Referring now to Table 1, the increasing streaming potential values show increasing deposition of lysozyme with increasing solution concentration. Surprisingly, a measurable change in streaming potential was realized with very low concentrations of lysozyme, demonstrating that very little of the protein is necessary for deposition on the hair; thus, the compositions of this invention may be made relatively economically.

### COMPARATIVE EXAMPLE 2

As a comparison to the compositions and methods of this invention, streaming potential analysis was conducted on blonde hair showing the effect on streaming potential of relatively high concentrations (0.5%) of various protein hydrolysates when applied to the hair. All treatment solutions in Example 2 were made with 0.5% of the specified protein hydrolysate in a solution of 1 mM KCl in deionized water. The results are shown in Table 2 below.

TABLE 2

| Protein | Source | ΔE |
|---|---|---|
| Keratin (Keratec IFP HMW) | Keratec Ltd., | 3.8 |
| | Lincoln, Canterbury, | |
| | NZ | |
| Soy | Croda Inc., Edison, | 4.9 ± 0.1 |
| | NJ | |
| Wheat | Croda Inc., Edison, | 5.3 ± 0.1 |
| | NJ | |
| Quaternized Wheat | Croda Inc., Edison, | 5.6 ± 1.0 |
| (Hydrotriticum WQ) | NJ | |

Referring now to Table 2, it can be seen that the streaming potential of these protein hydrolysates increased only slightly from untreated to treated hair. Thus, they do not significantly bind to hair and alter its streaming potential, even at the relatively high concentration of 0.5 wt. %.

### EXAMPLE 3

Lysozyme in various concentrations was adsorbed onto untreated blonde hair. Streaming potential was used to measure the extent of lysozyme binding to the hair. Portions of these treated hair samples were then subjected to the Lysozyme Activity Assay as described above for measurement of lysozyme activity at the various concentrations.

Referring now to FIG. 1, it can be seen that the fluorescence intensity, represented by the squares, increased with increasing lysozyme concentration. Likewise, the delta streaming potential, represented by the triangles, also increased with increasing concentration of lysozyme. Therefore, the lysozyme was deposited onto the hair surface, even at low concentrations, and the deposited lysozyme retained its antimicrobial activity.

### EXAMPLE 4

Streaming potential analysis was conducted on blonde hair showing the effect on streaming potential of a first treatment with a solution of lysozyme and a second treatment with a solution of sodium laureth sulfate (SLES) (available from Rhodia, Cranbury, N.J.). The solutions of 0.0125% lysozyme and 0.25% SLES were prepared and utilized at the respective concentrations in 1 mM KCl in deionized water.

Referring now to FIG. 2, the first five data points correspond to untreated hair, the next two data points correspond to hair after treatment with the lysozyme, the next four data points correspond to the hair after rinsing with 1 mM KCl solution, the next two data points correspond to the hair after treatment with SLES, and the last five data points correspond to the hair after rinsing with 1 mM KCl solution.

The data clearly show that the lysozyme was adsorbed onto the surface of the hair and remained even after subsequent rinsing cycles. The SLES treatment reduced the amount of lysozyme on the hair, but after rinsing to remove excess SLES (last five data points), the streaming potential increased again to about −10. This demonstrates that lysozyme remained bound to the hair surface even after treatment (washing) with the anionic surfactant SLES.

The specification and embodiments above are presented to aid in the complete and non-limiting understanding of the invention disclosed herein. Since many variations and embodiments of the invention can be made without departing from its spirit and scope, the invention resides in the claims hereinafter appended.

What is claimed is:

1. A cosmetic composition for providing a cosmetic benefit to a keratin-containing substrate comprising:
a) at least one naturally-occurring cationic protein having a biological function; and
b) a cosmetically acceptable carrier;
wherein said naturally-occurring cationic protein retains its essential configuration when in proximity to the keratin-containing substrate to preserve said biological function.

2. A cosmetic composition according to claim 1, wherein said naturally-occurring cationic protein has an Isoelectric Point of at least 6.

3. A cosmetic composition according to claim 2 wherein said naturally-occurring cationic protein has an Isoelectric Point of from about 8 to about 12.

4. A cosmetic composition according to claim 1, wherein said naturally-occurring cationic protein is selected from the group consisting of lysozyme, avidin, antimicrobial proteins, RNA or DNA binding proteins, proteases, methylated collagen, Cytochrome C, proteins involved in the aging process, Platelet Factor 4, protamine sulfate and mixtures thereof.

5. A method according to claim 4 wherein said antimicrobial proteins are selected from the group consisting of: magainin, defensins, cathelicdin and mixtures thereof.

6. A method according to claim 4 wherein said RNA or DNA binding proteins are selected from the group consisting of histones, ribonuclease A, Deoxyribonuclease and mixtures thereof.

7. A method according to claim 4 wherein said proteases are selected from the group consisting of Trypsin, Chymotrypsin, Papain, Caspase and mixtures thereof.

8. A method according to claim 4 wherein said protein involved in the aging process is +Telomerase.

9. A cosmetic composition according to claim 4, wherein said naturally-occurring cationic protein is lysozyme.

10. A cosmetic composition according to claim 4, wherein said naturally-occurring cationic protein is an avidin selected from the group consisting of streptavidin, cationic biotin binding proteins and mixtures thereof.

11. A cosmetic composition according to claim 1, wherein said biological function imparts a cosmetic benefit to said keratin-containing substrate.

12. A cosmetic composition according to claim 11, wherein said cosmetic benefit is selected from the group consisting of anti-aging, anti-microbial, antifungal, anti-inflammation, coloring, skin tightening, conditioning, cleansing, and the combinations thereof.

13. A cosmetic composition according to claim 1, wherein said naturally-occurring cationic protein has a concentration range from about 0.000001% to about 10% by weight.

14. A cosmetic composition according to claim 13, wherein said naturally-occurring cationic protein has a concentration range from about 0.001% to about 5% by weight.

15. A cosmetic composition according to claim 14, wherein said naturally-occurring cationic protein has a concentration range from about 0.01% to about 2% by weight.

16. A cosmetic composition according to claim 1, wherein said keratin-containing substrate is selected from the group consisting of hair, skin, nails, teeth, tissues, wool, and fur.

17. A cosmetic composition according to claim 16, wherein said keratin-containing substrate is hair.

18. A cosmetic composition according to claim 16, wherein said keratin-containing substrate is skin.

19. A method for providing a cosmetic benefit to a keratin-containing substrate comprising:
a) providing a cosmetic composition comprising at least one naturally-occurring cationic protein having a biological function; and a cosmetically acceptable carrier; and
b) applying said cosmetic composition to the keratin-containing substrate for a time period sufficient for said naturally-occurring cationic compound to be deposited on the substrate and form a layer;
wherein said naturally-occurring cationic protein retains its essential configuration when in proximity to the keratin-containing substrate to preserve said biological function.

20. A method according to claim 19, wherein said keratin-containing substrate is selected from the group consisting of hair, skin, nails, teeth, tissues, wool, and fur.

21. A method according to claim 19, wherein said biological function imparts a cosmetic benefit to the keratin-containing substrate.

22. A method according to claim 21, wherein said cosmetic benefit is selected from the group consisting of anti-aging, anti-microbial, antifungal, anti-inflammation, coloring, skin tightening, conditioning, cleansing, and the combinations thereof.

23. A method according to claim 19, wherein said cosmetic composition is applied to the keratin-containing substrate for from about 1 to about 20 minutes.

24. A method according to claim 23, wherein said cosmetic composition is applied to the keratin-containing substrate for from about 1 to about 10 minutes.

25. A method according to claim 24, wherein said cosmetic composition is applied to the keratin-containing substrate for from about 2 to about 5 minutes.

26. A method according to claim 19 further comprising a step of rinsing the cosmetic composition from the substrate with water.
